(19) United States
(12) Patent Application Publication
(10) Pub. No.: US 2007/0105782 A1
(43) Pub. Date: May 10, 2007

(54) Protectant Combinations for Reducing Toxicities

(75) Inventor: Kathleen C.M. Campbell, Glenarm, IL (US)

Correspondence Address: SENNIGER POWERS, ONE METROPOLITAN SQUARE, 16TH FLOOR, ST LOUIS, MO, 63102, US

(73) Assignee: BOARD OF TRUSTEES OF SOUTHERN ILLINOIS UNIVERSITY, Springfield, IL (US)

(21) Appl. No.: 11/539,975

(22) Filed: Oct. 10, 2006

Related U.S. Application Data
(60) Provisional application No. 60/725,082, filed on Oct. 7, 2005.

(51) Int. Cl. A61K 38/05; A61K 31/198; A61K 31/385; A61K 31/22

(52) U.S. Cl. 514/18; 514/546; 514/562; 514/440

(57) ABSTRACT

Pharmaceutical compositions and methods of preventing or reducing hearing or balance loss and damage to ear cells in patients who have been exposed to toxic levels of noise and other toxic insults are provided. These methods comprise administering an effective amount of a protectant combination or composition comprising two or more protectants selected from the group of methionine protectant agents, N-acetylcysteine, carnitine, magnesium ions, lipoic acid, ebselen, glutathione, and glutathione ester. These protectant combinations can be administered prior to, simultaneously with, or subsequently to exposure to noise other toxic insults.

### REFERENCE TO RELATED APPLICATIONS

This application claims the benefit of U.S. provisional application Ser. No. 60/725,082, filed on Oct. 7, 2005.

### FIELD OF THE INVENTION

The present invention generally relates to protectant combinations useful for treating the conditions of sudden hearing loss and autoimmune inner ear disorder and various harmful effects of noise, platinum-coordination compounds, aminoglycoside antibiotics, radiation, loop diuretics, iron chelating agents, and quinine and quinidine.

### BACKGROUND

Approximately 30 million people in the U.S. suffer from sensorineural hearing loss. The potential treatment pool for this condition includes workers in factories, construction operations, communications, the military, and the airline industry. People working in an environment with damaging noise or toxins would potentially benefit from an effective method of treatment. In addition, individuals receiving toxic medications (e.g., cancer chemotherapy, aminoglycoside antibiotics, loop diuretics, etc.) can suffer sensorineural hearing loss. Further, patients suffering from idiopathic hearing loss and autoimmune inner ear disease also need effective treatments. Thus, a need exists for more effective agents for protection from side effects resulting from exposure to noise or toxic insults.

### SUMMARY OF THE INVENTION

Among the various aspects of the present invention is a protectant combination and a protectant compositions and methods for their use. One aspect of the invention is a therapeutic combination or therapeutic composition comprising two or more protectant agents selected from a group consisting of methionine protectant agents, N-acetylcysteine, carnitine, magnesium ions, lipoic acid, ebselen, glutathione, and glutathione ester. The methionine protectant agent corresponds to Formula 1 described below and at least one of said protectant agents is present in a concentration of at least about 10 wt. % based on the total weight of active ingredients.

Various combinations of protectant agents are described in more detail below in Table 1. The protectant combinations can also comprise compositions, particularly pharmaceutical compositions. Further, each of the combinations or compositions detailed in Table 1 can be combined to form a composition comprising the protectant agents designated and a toxic insult selected from the group of platinum-coordination compounds, aminoglycoside antibiotics, loop diuretics, iron chelating agents, quinine and quinidine, and combinations thereof.

Other aspects are methods to treat a variety of toxicities (e.g., ototoxicity, nephrotoxicity, neurotoxicity, alopecia, gastrointestinal disorder, or reduced survival) resulting from administration of a chemotherapeutic effective amount of an anti-tumor platinum-coordination compound, an aminoglycoside antibiotic or resulting from exposure to noise or radiation by administering the therapeutic protectant combinations and compositions of the invention.

Yet other aspects are methods to treat sudden hearing loss and autoimmune inner ear disease by administering the therapeutic protectant combinations and compositions of the invention.

Other objects and features will be in part apparent and in part pointed out hereinafter.

### DETAILED DESCRIPTION OF THE INVENTION

The present invention is directed to protectant combinations useful for treating conditions and side effects from administration of agents that involve cell damage. One mechanism for this cell damage is understood to be an oxidative process. Cell damage by oxidative processes can occur in sudden hearing loss and autoimmune inner ear disorder and from the effects of noise and radiation exposure, as well as administration of platinum-coordination compounds, aminoglycoside antibiotics, loop diuretics, iron chelating agents, and quinine and quinidine. This cell damage can be treated by administration of the protectant combinations of two or more protective agents selected from the group consisting of methionine protectant agents, N-acetylcysteine, carnitine, magnesium ions, lipoic acid, ebselen, glutathione, and glutathione ester.

The protectant combinations and compositions of the invention have a variety of mechanisms of action to treat cell damage resulting from the insults described above. These combinations include complementary agents that by various mechanisms produce the prophylactic and/or therapeutic effects of neuroprotection and preventing or ameliorating both temporary and permanent noise induced hearing loss. Some of the ways these effects are understood to be accomplished are by (1) reducing oxidative stress to the cell by containing more than one type of free radical scavenger; (2) increasing both cytosolic and mitochondrial glutathione levels; (3) enhancing and preserving mitochondrial function other than glutathione levels; and (4) enhancing blood flow and cochlear oxygen levels.

Accordingly, the protectant combinations of the invention can be used as otoprotectants, neuroprotectants, gastrointestinal protectants, weight loss protectants, alopecia protectants, and survival-enhancing agents. In various preferred embodiments, the protectant combinations are used as otoprotectants and neuroprotectants. Each protectant is described in more detail below.

Protectant Combinations

As described above, the protectant combinations or compositions of the invention generally comprise two or more protectant agents selected from the group consisting of methionine protectant agents, N-acetylcysteine, carnitine, magnesium ions, lipoic acid, ebselen, and glutathione ester. Generally, the protectant combinations and compositions can contain the following protectant agents (described in Table 1) in the concentrations and proportions described below. The methionine protectant agent is an agent having a structure of Formula 1 that is not methionine. The protectant agents comprising each combination or combination are marked with an “X.”

| Combination | | Methionine | | | | | | |
|---|---|---|---|---|---|---|---|---|
| or | | protectant | N-acetyl- | | Magnesium | Lipoic | | Glutathione |
| composition | Methionine | agent | cysteine | Carnitine | ions | acid | Ebselen | ester |
| 1 | X | X | | | | | | |
| 2 | X | | X | | | | | |
| 3 | X | | | X | | | | |
| 4 | X | | | | X | | | |
| 5 | X | | | | | X | | |
| 6 | X | | | | | | X | |
| 7 | X | | | | | | | X |
| 8 | | X | X | | | | | |
| 9 | | X | | X | | | | |
| 10 | | X | | | X | | | |
| 11 | | X | | | | X | | |
| 12 | | X | | | | | X | |
| 13 | | X | | | | | | X |
| 14 | | | X | X | | | | |
| 15 | | | X | | X | | | |
| 16 | | | X | | | X | | |
| 17 | | | X | | | | X | |
| 18 | | | X | | | | | X |
| 19 | | | | X | X | | | |
| 20 | | | | X | | X | | |
| 21 | | | | X | | | X | |
| 22 | | | | X | | | | X |
| 23 | | | | | X | X | | |
| 24 | | | | | X | | X | |
| 25 | | | | | X | | | X |
| 26 | | | | | | X | X | |
| 27 | | | | | | X | | X |
| 28 | | | | | | | X | X |
| 29 | X | X | X | | | | | |
| 30 | X | X | | X | | | | |
| 31 | X | X | | | X | | | |
| 32 | X | X | | | | X | | |
| 33 | X | X | | | | | X | |
| 34 | X | X | | | | | | X |
| 35 | X | | X | X | | | | |
| 36 | X | | X | | X | | | |
| 37 | X | | X | | | X | | |
| 38 | X | | X | | | | X | |
| 39 | X | | X | | | | | X |
| 40 | X | | | | | | | |
| 41 | X | | | X | X | | | |
| 42 | X | | | X | | X | | |
| 43 | X | | | X | | | X | |
| 44 | X | | | X | | | | X |
| 45 | X | | | | X | X | | |
| 46 | X | | | | X | | X | |
| 47 | X | | | | X | | | X |
| 48 | X | | | | | X | X | |
| 49 | X | | | | | X | | X |
| 50 | X | | | | | | X | X |
| 51 | | X | X | X | | | | |
| 52 | | X | X | | X | | | |
| 53 | | X | X | | | X | | |
| 54 | | X | X | | | | X | |
| 55 | | X | X | | | | | X |
| 56 | | X | | X | X | | | |
| 57 | | X | | X | | X | | |
| 58 | | X | | X | | | X | |
| 59 | | X | | X | | | | X |
| 60 | | X | | | X | X | | |
| 61 | | X | | | X | | X | |
| 62 | | X | | | X | | | X |
| 63 | | X | | | | X | X | |
| 64 | | X | | | | X | | X |
| 65 | | X | | | | | X | X |
| 66 | | | X | X | X | | | |
| 67 | | | X | X | | X | | |
| 68 | | | X | X | | | X | |
| 69 | | | X | X | | | | X |
| 70 | | | X | | X | X | | |
| 71 | | | X | | X | | X | |
| 72 | | | X | | X | | | X |
| 73 | | | X | | | X | X | |
| 74 | | | X | | | X | | X |
| 75 | | | X | | | | X | X |
| 76 | | | | X | X | X | | |
| 77 | | | | X | X | | X | |
| 78 | | | | X | X | | | X |
| 79 | | | | X | | X | X | |
| 80 | | | | X | | X | | X |
| 81 | | | | X | | | X | X |
| 82 | | | | | X | X | X | |
| 83 | | | | | X | X | | X |
| 84 | | | | | | X | X | X |
| 85 | X | X | X | X | | | | |
| 86 | X | X | X | | | | | |
| 87 | | | | | X | | | |
| 88 | X | X | X | | | X | | |
| 89 | X | X | X | | | | X | |
| 90 | X | X | X | | | | | X |
| 91 | X | | X | X | X | | | |
| 92 | X | | X | X | | X | | |
| 93 | X | | X | X | | | X | |
| 94 | X | | X | X | | | | X |
| 95 | X | | | X | X | X | | |
| 96 | X | | | X | X | | X | |
| 97 | X | | | X | X | | | X |
| 98 | X | | | | X | X | X | |
| 99 | X | | | | X | X | | X |
| 100 | X | | | | | X | X | X |

A. Methionine

Methionine or a methionine-like compound can be one component of the protectant combinations of the present invention. It is one of the few amino acids that are reversibly oxidized. Because it can be reversibly oxidized, it is an effective free radical scavenger. As a free radical scavenger, methionine can react to reduce a free radical species and thus form a methionine sulfoxide. In reacting with the free radical, the methionine neutralizes an oxidant molecule that would otherwise react with another cellular molecule and potentially cause cell damage by the oxidation of species important for cell function. Due to its antioxidant properties, methionine can maintain the ratio of reduced glutathione to oxidized glutathione in a cell undergoing oxidative stress.

Another function of methionine is as a source of cysteine for glutathione synthesis. Glutathione plays an essential role in certain anti-oxidant pathways as it can react with an oxidant to neutralize and thus prevent potential damage to cells from reaction of the oxidant with cell molecules other than glutathione. Methionine can be converted by a cell to glutathione in a number of enzyme-mediated steps. The preservation of the protectant system including glutathione may be important for preventing and treating cell damage from oxidants. In addition to being a precursor for glutathione, methionine prevents efflux of glutathione from an injured cell; this action reduces the damage to the injured cell by supporting the protectant system and cell repair mechanisms. Further, methionine promotes glutathione formation within the mitochondria. Mitochondrial injury caused by excessive free radical generation within the mitochondria can play a key role in cell death. Inhibition of mitochondrial self-repair enhances free radical damage while support of mitochondrial self-repair through support of the protectant pathways reduces cellular damage from free radicals.

Analogs or derivatives of methionine useful in the present invention are compounds containing a methionine moiety, or a methionine-like moiety including a thioether group, that exhibit an otoprotectant effect, a weight-loss protectant effect, a gastrointestinal protectant effect, a neuroprotectant effect, an alopecia protectant effect, and/or a survival-enhancing effect when used in conjunction with an antitumor platinum coordination compound administered in an effective chemotherapeutic dose, a loop diuretic compound, an aminoglycoside antibiotic, an iron chelator, quinine, or in conjunction with exposure to noise or radiation. Among the compounds structurally related to D-Met that can be employed in the present invention are those containing the C—S—C— (thioether) moiety. These include, but are not limited to, compounds corresponding to Formula 1: 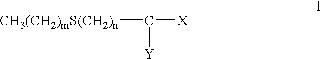
wherein m is an integer from 0 to 3; n is an integer from 1 to 3; X═—OR¹, —OCOR¹, —COOR¹, —CHO, —CH(OR¹)₂, or —CH₂OH; Y═—NR²R³ or —OH; R¹═H or a substituted or unsubstituted, straight, branched chain, or cyclic alkyl group having 1 to 6 carbon atoms, preferably 1 to 4 carbon atoms; R²═H or a substituted or unsubstituted, straight or branched chain acyl group having 1 to 6 carbon atoms, preferably 1 to 4 carbon atoms; and R³═H or a substituted or unsubstituted, straight or branched chain acyl group having 1 to 6 carbon atoms, preferably 1 to 4 carbon atoms; or a pharmaceutically acceptable salt thereof.

Non-limiting examples of such methionine protectant agents include D-methionine (D-Met), L-methionine, a mixture of D-methionine and L-methionine, normethionine, homomethionine, methioninol, hydroxy methionine, ethionine, or pharmaceutically acceptable salts thereof. S-adenosyl-L-methionine, or a pharmaceutically acceptable salt thereof, can also be employed. Methionine protectant agents of the present invention can be in the D-, L-, or DL- form, and include pharmaceutically acceptable N-(mono- and dicarboxylic acid) acyl derivatives and alkyl esters thereof. Exemplary acyl derivatives include the formyl, acetyl, propionyl, and succinyl derivatives. Exemplary ester derivatives include the methyl, ethyl, propyl, isopropyl, and butyl esters.

Collectively, methionine, along with the other compounds discussed above, can be referred to as “methionine protectant agents.”

In many of the various embodiments, methionine can be present either as a racemic mixture, predominantly as the L-isomer, or predominantly as the D-isomer. Optically active D-methionine is preferred. When, for example, the methionine is present predominantly as the D-isomer, that means that at least about 60%, or about 75%, or about 90% or about 98% of the total methionine is made up of the D-isomer.

In many preferred embodiments, methionine or methionine protectant agent is present in a concentration of at least about 1 wt. %, or about 4 wt. %, or about 6 wt. %, or about 8 wt. %, or about 10 wt. %, or about 15 wt. %, or about 20 wt. %, or about 25 wt. % based on the total weight of active ingredients. Typically, methionine or methionine protectant agent is present in a concentration of up to about 80 wt. %, or about 85 wt. %, or about 90 wt. %, or about 95 wt. % based on the total weight of active ingredients. When D-methionine has a 70% enantiomeric excess (“e.e.”), that is, 85 weight or mole percent of one enantiomer and 15 weight or mole percent of the other enantiomer. The D-methionine can be in any form suitable for use in the present invention, including pharmaceutically acceptable salts, e.g., the chloride, iodide, dicyclohexylamine, dicyclohexylammonium, cyclohexylammonium, cyclohexylamine, sulfonate, and acetate salts.

Most human studies using methionine at moderate dosages have reported no side effects. Methionine toxicity can result from very high dosing of racemic or L-methionine, particularly in the presence of a low protein diet and/or in developing animals as opposed to adults. However, in the studies that found a toxic effect, the methionine doses were much higher than anticipated human therapeutic levels as administered in accordance with the method of the invention.

Further, D-methionine is substantially safer than L-methionine, and several studies suggest that D-methionine itself is not toxic unless it is converted to the L-isomer. In the human, D-methionine results in higher plasma levels than L-methionine, which may be advantageous for a protective agent. In humans, 60-70% of D-methionine is excreted without conversion to the L-isomer except in L-methionine deprivation; L-methionine deprivation can increase the conversion of D-methionine to L-methionine above the normal level.

Methionine may be included in the protectant combination so that upon administration of the combination, methionine is administered at a dose of about 10 mg/kg to about 500 mg/kg; preferably, from about 50 mg/kg to about 400 mg/kg; more preferably, from about 100 mg/kg to about 250 mg/kg. As described above, the methionine dose may be provided by a racemic mixture of D-methionine and L-methionine, by a source predominantly comprising L-methionine, or, preferably, by a source predominantly comprising D-methionine.

In therapeutic combinations of the invention that comprise methionine, the proportion of the methionine to the total amount of other protectants in the combination is from about 1:100 to 100:1; preferably, from about 1:10 to about 10:1; more preferably, from about 1:5 to 5:1; even more preferably, from about 1:1 to 3:1.

B. Carnitine

Carnitine protectant agents comprise carnitine, acetylcarnitine, and propionylcarnitine. In various embodiments, acetylcarnitine may be a component of the protectant combinations and compositions. Acetylcarnitine reduces stress induced mitochondrial damage and enhances repair. It does this by enhancing cellular energy utilization, enhancing biogenesis, maintaining cardiolipin (a key mitochondrial membrane component), and maintaining mitochondrial electron transport while the cell undergoes oxidative stress. Each of these actions is or can be important to reducing cell damage and supporting repair of the cell after such damage. Complementary to the above actions, acetylcarnitine enhances cytosolic glutathione synthesis, but not mitochondrial glutathione levels.

In various embodiments, the acetylcarnitine comprises acetyl-L-carnitine, acetyl-D-carnitine and combinations thereof. Preferably, the acetylcarnitine comprises acetyl-L-carnitine.

In various embodiments, acetylcarnitine may be present in a concentration of at least about 1 wt. %, or about 4 wt. %, or about 6 wt. %, or about 8 wt. %, or about 10 wt. %, or 15 wt. %, or 20 wt. %, or 25 wt. % based on the total weight of active ingredients. Typically, acetylcarnitine is present in a concentration of up to about 80 wt. %, or about 85 wt. %, or about 90 wt. %, or about 95 wt. % based on the total weight of active ingredients.

There are no known toxic side effects of acetylcarnitine administration at the dosages described. Acetylcarnitine is included in the protectant combination so that upon administration of the combination, the dose of acetylcarnitine is from about 20 mg/kg to about 400 mg/kg; preferably, from about 40 mg/kg to about 200 mg/kg; even more preferably, from about 50 mg/kg to about 150 mg/kg.

In therapeutic combinations of the invention which include acetylcarnitine, the proportion of acetylcarnitine to the total amount of other protectants in the combination is from about 1:100 to 1:1; preferably, from about 1:50 to about 1:1; more preferably, from about 1:25 to 1:1; even more preferably, from about 1:10 to 1:1.

C. N-acetylcysteine

N-acetylcysteine (NAC) can be a component of the protectant combinations. NAC acts as a free radical scavenger similar to methionine as described above. In addition to acting as a free radical scavenger, NAC can act as a precursor for glutathione synthesis in the cytosolic fluid of a cell, but unlike methionine does not activate glutathione synthesis in the mitochondria.

In addition, to the above effects, NAC may act as a neuroprotectant. In this case, a neuroprotectant is an agent that protects against neurotoxicity, which can be exhibited by detrimental or pathologic changes in the structure or function in the neurologic system or any part thereof. Further to these effects, NAC prevents activation of the c-Jun N-terminal kinases (JNK) pathway. Members of the JNK family regulate signal transduction in response to environmental stress and other insults. Such an activation of the JNK family can lead to cell death.

NAC can cause nausea and vomiting at high doses. Cysteine stone formation has been reported in rare cases and is generally associated with patient dehydration.

In many preferred embodiments, NAC is present in a concentration of at least about 1 wt. %, or about 2 wt. %, or about 3 wt. %, or about 4 wt. %, or about 5 wt. %, or about 10 wt. %, or about 15 wt. %, or about 25 wt. % based on the total weight of active ingredients. Typically, NAC is present in a concentration of up to about 80 wt. %, or about 85 wt. %, or about 90 wt. %, or about 95 wt. % based on the total weight of active ingredients.

NAC may be included in the protectant combination so that upon administration of the combination, the dose of NAC is from about 20 mg/kg to about 400 mg/kg; preferably, from about 20 mg/kg to about 200 mg/kg; even more preferably, from about 40 mg/kg to about 150 mg/kg. In therapeutic combinations of the invention which include NAC, the proportion of NAC to the total amount of other protectants in the combination is from about 1:100 to 1:1; preferably, from about 1:50 to about 1:1; more preferably, from about 1:25 to 1:1; even more preferably, from about 1:10 to 1:1.

D. Magnesium Ions

Magnesium ions can be a component of the protectant combinations of the invention. Magnesium ions reduce noise-induced temporary threshold shift and permanent threshold shift. This is important because many of the other protectants are known to reduce permanent threshold shifts induced by noise, but not temporary threshold shift. It is hypothesized that the reduction of temporary threshold shifts is due to increased cochlear blood flow and increased partial oxygen pressure in the cochlea. Greater concentrations of magnesium ions provide needed energy to cochlea cells that has been depleted by the cell's response to the toxic insult. If this energy is not restored, cell damage and potential cell death could occur. In addition to these effects, magnesium ions may counteract noise induced hypertension.

Magnesium ions can be provided by a variety of compounds. For example, the source of magnesium ions can be selected from the group consisting of magnesium aspartate, magnesium sulfate, magnesium chloride, magnesium oxide, magnesium gluconate, magnesium citrate, magnesium hydroxide, magnesium orotate, magnesium arginate, and combinations thereof.

In many preferred embodiments, magnesium ions are present in a concentration of at least about 1 wt. %, or about 2 wt. %, or about 3 wt. %, or about 4 wt. %, or about 5 wt. %, or about 10 wt. %, or about 15 wt. %, or about 25 wt. % based on the total weight of active ingredients. Typically, magnesium ions are present in a concentration of up to about 80 wt. %, or about 85 wt. %, or about 90 wt. %, or about 95 wt. % based on the total weight of active ingredients.

Magnesium ions can cause diarrhea and other gastrointestinal symptoms at high doses. Magnesium ions are preferably included in the protectant combination in such proportion and dosage that upon administration of the combination, the dose of magnesium ions on an Mg²⁺ weight basis is from about 20 mg/kg to about 200 mg/kg; more preferably from about 20 mg/kg to about 100 mg/kg; and still more preferably from about 30 mg/kg to about 80 mg/kg.

In therapeutic combinations of the invention which include magnesium ions, the proportion of the magnesium ions to the total amount of other protectants in the combination is from about 1:100 to 100: 1; preferably, from about 1:10 to about 10: 1; more preferably, from about 1:5 to 5:1; even more preferably, from about 1:3 to 1:1.

E. Lipoic Acid

Lipoic acid can be a component of the protectant combinations of the invention. Lipoic acid is a powerful antioxidant and along with its reduced form, dihydrolipoic acid, can scavenge many reactive oxygen species. For example, both lipoic acid and dihydrolipoic acid can scavenge hydroxy radicals, nitric oxide radicals, peroxynitrites, hydrogen peroxide and hypochlorite, whereas lipoic acid can also scavenge singlet oxygen and dihydrolipoic acid can scavenge superoxide and peroxyl reactive oxygen species.

In many preferred embodiments, lipoic acid may be present in a concentration of at least about 1 wt. %, or about 2 wt. %, or about 3 wt. %, or about 4 wt. %, or about 5 wt. %, or about 10 wt. %, or about 15 wt. %, or about 25 wt. % based on the total weight of active ingredients. Typically, lipoic acid is present in a concentration of up to about 80 wt. %, or about 85 wt. %, or about 90 wt. %, or about 95 wt. % based on the total weight of active ingredients.

Lipoic acid has no known toxic side effects at the dosage used in the protectant combinations. Lipoic acid may be included in the protectant combination so that upon administration of the combination, the dose of lipoic acid is from about 0.1 mg/kg to about 10 mg/kg; preferably, from about 0.2 mg/kg to about 5 mg/kg; even more preferably, from about 0.2 mg/kg to about 3 mg/kg.

The proportion of lipoic acid to the total amount of other protectants in the combination is from about 1:100 to 1:1; preferably, from about 1:50 to about 1:1; more preferably, from about 1:25 to 1:1; even more preferably, from about 1:10 to 1:1.

F. Ebselen

Ebselen can be a component of the protectant combinations of the invention. Ebselen has the chemical structure that follows. 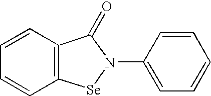
Ebselen is an antioxidant compound that has been shown to be a neuroprotective agent. In addition to its antioxidant activity, ebselen acts as a glutathione peroxidase mimic and thus plays a role in antioxidant pathways in cells. These functions aid ebselen's action as an inhibitor of free radical induced apoptosis.

In many preferred embodiments, ebselen may be present in a concentration of at least about 1 wt. %, or about 2 wt. %, or about 3 wt. %, or about 4 wt. %, or about 5 wt. %, or about 10 wt. %, or about 15 wt. %, or about 25 wt. % based on the total weight of active ingredients. Typically, ebselen is present in a concentration of up to about 80 wt. %, or about 85 wt. %, or about 90 wt. %, or about 95 wt. % based on the total weight of active ingredients.

G. Glutathione and Glutathione Ester

Glutathione and glutathione esters can be components of the protectant combinations and compositions of the invention. In preferred embodiments, the monoethyl and monoiospropyl esters of glutathione are components of the protectant combinations and compositions. Glutathione esters are understood to easily cross the cell wall to interact with cell contents and provide a protective effect against various deleterious oxidative processes.

In many preferred embodiments, glutathione ester (either monoethyl ester or monoisopropyl ester) may be included in the combinations and compositions so that upon administration of the combination, the dose of glutathione ester is from about 0.001 mg/kg to about 500 mg/kg; preferably, from about 0.01 mg/kg to about 250 mg/kg; even more preferably, from about 1 mg/kg to about 200 mg/kg.

The typical and preferred ranges and proportions for a combination of methionine and acetylcarnitine in the protectant combinations of the invention are as follows. The methionine can typically be present in the combination from about 10 wt. % to about 90 wt. %; preferably, from about 25 wt. % to about 75 wt. %; more preferably, from about 40 wt. % to about 60 wt. % based on the total weight of active ingredients. The acetylcarnitine can typically be present in the combination from about 10 wt. % to about 90 wt. %; preferably, from about 25 wt. % to about 75 wt. %; more preferably, from about 40 wt. % to about 60 wt. % based on the total weight of active ingredients. The ratio of methionine to acetyl carnitine can generally range from about 1:9 to about 9:1; preferably, from about 1:3 to about 3:1; more preferably, from about 1:1.5 to about 1.5:1.

The typical and preferred ranges and proportions for a combination of methionine and magnesium ions in the protectant combinations of the invention are as follows. The methionine can typically be present in the combination from about 10 wt. % to about 90 wt. %; preferably, from about 25 wt. % to about 75 wt. %; more preferably, from about 40 wt. % to about 75 wt. % based on the total weight of active ingredients. The magnesium ions can typically be present in the combination from about 10 wt. % to about 90 wt. %; preferably, from about 25 wt. % to about 75 wt. %; more preferably, from about 25 wt. % to about 60 wt. % based on the total weight of active ingredients. The ratio of methionine to magnesium ions can generally range from about 1:9 to about 9:1; preferably, from about 1:3 to about 3:1; more preferably, from about 1:1 to about 3:1.

The typical and preferred ranges and proportions for a combination of acetylcarnitine and magnesium ions in the protectant combinations of the invention are as follows. The acetylcarnitine can typically be present in the combination from about 10 wt. % to about 90 wt. %; preferably, from about 25 wt. % to about 75 wt. %; more preferably, from about 40 wt. % to about 75 wt. % based on the total weight of active ingredients. The magnesium ions can typically be present in the combination from about 10 wt. % to about 90 wt. %; preferably, from about 25 wt. % to about 75 wt. %; more preferably, from about 25 wt. % to about 60 wt. % based on the total weight of active ingredients. The ratio of acetylcarnitine to magnesium ions can generally range from about 1:9 to about 9:1; preferably, from about 1:3 to about 3:1; more preferably, from about 1:1 to about 3:1.

The typical and preferred ranges and proportions for a combination of methionine and N-acetylcysteine in the protectant combinations of the invention are as follows. The methionine can typically be present in the combination from about 10 wt. % to about 90 wt. %; preferably, from about 25 wt. % to about 75 wt. %; more preferably, from about 40 wt. % to about 75 wt. % based on the total weight of active ingredients. The N-acetylcysteine can typically be present in the combination from about 10 wt. % to about 90 wt. %; preferably, from about 25 wt. % to about 75 wt. %; more preferably, from about 25 wt. % to about 60 wt. % based on the total weight of active ingredients. The ratio of methionine to N-acetylcysteine can generally range from about 1:9 to about 9:1; preferably, from about 1:3 to about 3:1; more preferably, from about 1:1 to about 3:1.

The typical and preferred ranges and proportions for a combination of acetylcarnitine and N-acetylcysteine in the protectant combinations of the invention are as follows. The acetylcarnitine can typically be present in the combination from about 10 wt. % to about 90 wt. %; preferably, from about 25 wt. % to about 75 wt. %; more preferably, from about 40 wt. % to about 75 wt. % based on the total weight of active ingredients. The N-acetylcysteine can typically be present in the combination from about 10 wt. % to about 90 wt. %; preferably, from about 25 wt. % to about 75 wt. %; more preferably, from about 25 wt. % to about 60 wt. % based on the total weight of active ingredients. The ratio of acetylcarnitine to N-acetylcysteine can generally range from about 1:9 to about 9:1; preferably, from about 1:3 to about 3:1; more preferably, from about 1:1 to about 3:1.

The typical and preferred ranges for a combination of methionine, acetylcarnitine and magnesium ions in the protectant combinations of the invention are as follows. The methionine can typically be present in the combination from about 10 wt. % to about 80 wt. %; preferably, from about 25 wt. % to about 65 wt. %; more preferably, from about 30 wt. % to about 50 wt. % based on the total weight of active ingredients. The acetylcarnitine can typically be present in the combination from about 10 wt. % to about 80 wt. %; preferably, from about 25 wt. % to about 65 wt. %; more preferably, from about 30 wt. % to about 50 wt. % based on the total weight of active ingredients. The magnesium ions can typically be present in the combination from about 10 wt. % to about 50 wt. %; preferably, from about 10 wt. % to about 40 wt. %; more preferably, from about 10 wt. % to about 30 wt. % based on the total weight of active ingredients.

The typical and preferred ranges for a combination of methionine, acetylcarnitine and N-acetylcysteine in the protectant combinations of the invention are as follows. The methionine can typically be present in the combination from about 10 wt. % to about 80 wt. %; preferably, from about 25 wt. % to about 65 wt. %; more preferably, from about 30 wt. % to about 50 wt. % based on the total weight of active ingredients. The acetylcarnitine can typically be present in the combination from about 10 wt. % to about 80 wt. %; preferably, from about 25 wt. % to about 65 wt. %; more preferably, from about 30 wt. % to about 50 wt. % based on the total weight of active ingredients. The N-acetylcysteine can typically be present in the combination from about 10 wt. % to about 50 wt. %; preferably, from about 10 wt. % to about 40 wt. %; more preferably, from about 10 wt. % to about 30 wt. % based on the total weight of active ingredients.

H. Combinations of Protectant Agents and Toxins

The protectant combinations and compositions of the invention can be further combined to form combinations and compositions comprising any of the combinations and compositions described in Table 1 and a toxic insult selected from the group of platinum-coordination compounds, aminoglycoside antibiotics, loop diuretics, iron chelating agents, quinine and quinidine, and combinations thereof. The protectant combinations and compositions are described in detail above and the toxic insults are described in more detail below.

In some of the various embodiments, each of the toxic insults can be added to the combinations and compositions described in Table 1. In many of these embodiments, agents that serve to improve the compatibility of the various components may be advantageous. For example, addition of various carriers, fillers, surface active agents, suspending agents, etc. to the composition may improve the physical characteristics of the composition.

Therapeutic Applications of Protectant Combinations

The protectant combinations described above have many advantages as described below. In addition, the toxic insults, the administration routes and forms and the dosing protocol are discussed in more detail below.

A. Advantages of Combinations

There are a variety of advantageous aspects of the protectant combinations of the present invention. One advantage of the protectant combinations is that multiple free radical scavengers and antioxidants are acting on the affected cells. For example, one type of free radical scavenger may be reactive with a particular oxidative species while not being reactive with other oxidative species produced from another type of toxic insult on the cell. In this case, one free radical scavenger may be particularly reactive with free radicals generated by a particular insult (e.g., free radicals generated from noise exposure) and thus be rapidly depleted while another free radical scavenger may not be as reactive with free radicals generated by that insult and thus be preserved at a higher concentration to combat other types of free radicals. In the situation described above, the overall protection from oxidative species is increased with more than one type of free radical scavenger as each type has a different reactivity with different oxidative species.

In addition to the differences in reactivity with free radicals, the protectant combinations of the invention provide increased energy resources and increased blood flow to the cochlea. This increased energy and blood flow reduces cochlea damage by providing energy to the cochlea cells that are depleted by toxic insults such as noise. Without the added energy to aid the cell's repair mechanisms, the cell could be permanently damaged or die. Another advantageous result of this additional energy is protection from temporary threshold shift.

Further to the mechanisms described above, the protectant combinations of the present invention provide increased levels of cytosolic and mitochondrial glutathione. This is important as many single protectants can provide increased levels of glutathione in either the cytosol or mitochondria, but not in both.

Finally, even though the protectant combinations of the present invention have few side effects at the doses applied, the combination allows reduction in the dose of one or more protectant(s) relative to the dosage that would be required if each such protectant were used alone. Such relative reduction in dosage of a given protectant is compensated for by concomitantly increasing the dose of another agent.

B. Toxic Insults

1. Noise

Noise-induced hearing loss, both impulse and chronic exposure, can damage hearing. In impulse noise, including blast exposure, the patient may suffer significant tympanic membrane and middle ear damage in addition to the cochlear and neural damage that typically occurs with toxic noise exposure. In chronic exposure, which generally occurs at lower intensity levels, middle ear and tympanic membrane damage are unlikely, but cochlear and neural damage are very likely. In noise exposure, the primary and initial damage is generally cochlear, with secondary neural degeneration of the auditory system occurring over time. Noise-induced hearing loss has been reviewed by the present inventor in the book entitled Essential Audiology For Physicians (1998) Singular Publishing Group, Inc., San Diego.

Noise-induced hearing loss can occur secondary to a single very loud noise exposure, or secondary to relatively high-level noise exposure over a long period of time. The risk of noise-induced hearing loss is related to both sound intensity and duration. Both the Occupational Safety and Health Administration (OSHA) and the Environmental Protection Agency (EPA) have established standards relevant to noise exposure levels in industry. The OSHA Permissible Noise Exposure Levels range from a duration of 32 hours at a sound level of 80 dBA to 0.25 hours at 115 dBA. For every 5 dB increase in the noise level, the allowable exposure duration is halved. Non-industrial, e.g., recreational, noises intense enough to damage hearing can vary, for example, from approximately 90 dBA (lawnmower) to approximately 140 dBA (shotgun blast).

2. Platinum-Coordination Compounds

Cisplatin (CDDP; cis-diamminedichloro-platinum(II)) is currently the anti-tumor platinum coordination compound most frequently employed in the therapy of testicular cancer, ovarian tumors, and a variety of other cancers. Methods of employing CDDP clinically are well known in the art (Nicolini, 1987). For example, CDDP can be administered in a single day over a six hour period, once per month, by slow intravenous infusion. For localized lesions, CDDP can be administered by local injection. Intraperitoneal infusion can also be employed. CDDP can be administered in doses as low as 10 mg/m² per treatment if part of a multi-drug regimen, or if the patient has an adverse reaction to higher dosing. At the low end, a more common clinical dose is about 30 mg/m²; the high end of the range is about 120 to about 150 mg/m² per treatment. When used in conjunction with D-Met or other methionine protective agents, these dosages can be increased.

CDDP is representative of a broad class of water-soluble, platinum coordination compounds well known in the art that provide platinum in the form of an ion having anti-tumor activity. Among the anti-tumor platinum coordination compounds described in the literature which are useful in the methods of the present invention are, for example, trans-diaminedichloro-platinum(II), cis-diamine-diaquaplatinum(II)-ion, cis-diaminedichloroplatinum(II)-ion, chloro(diethylenetriamine)-platinum(II) chloride, dichloro(ethylenediamine)-platinum(lI), diamine(1,1-cyclobutanedicarboxylato)-platinum(II) (carboplatin), spiroplatin, dichlorotrans-dihydroxybisisopropolamine platinum IV (iproplatin), diamine(2-ethylmalonato)-platinum(II), ethylenediamine-malonatoplatinum(II), aqua(1,2-diaminodiclohexane)-sulfatoplatinum(II), (1,2-diaminocyclohexane)malonato-platinum(II), (4-carboxyphthalato)(1,2-diaminocyclo-hexane)-platinum(II), (1,2-diaminocyclohexane)-(isocitrato)platinum(II), (1,2-diaminocyclohexane)-cis(pyruvato)platinum(II), and (1,2-diaminocyclohexane)-oxalatoplatinum(II).

3. Aminoglycoside Antibiotics

The aminoglycoside antibiotics share several structural features: they each contain one or more sugar moieties and a streptidine ring, and they each have one or more amino or guanidino groups. The currently available aminoglycoside antibiotics include streptomycin, kanamycin, gentamicin, amikacin, neomycin, netilmicin, paromomycin, vancomycin, hygromycin, erythromycin and tobramycin. One of the principal dangers associated with the use of aminoglycoside antibiotics is their ototoxicity, which is associated with either hearing loss (cochlear damage), vertigo (vestibular damage), or both. An early sign is tinnitus accompanied by loss of high-frequency hearing. Early detection of hearing loss can be reversed; prolonged treatment results in permanent hearing loss. The concomitant administration of other drugs that cause similar adverse effects potentiates the adverse effects of the aminoglycosides. Such other drugs include loop diuretic agents, as discussed below.

4. Radiation

Exposure to radiation, whether intentional, as in radiation therapy, or unintentional, as by accident, war, etc., can result in ototoxicity, as well as neural damage (neurotoxicity), alopecia, gastrointestinal disorders, and reduced patient survival. Although physical rather than chemical, radiation can be considered another “ototoxin” in view of its toxicity to the ear and hearing. Radiation-induced hearing loss is more likely to involve the middle ear than is hearing loss caused by platinum-containing compounds or loop diuretics; however, cochlear and neural problems can also occur.

Radiation-induced ototoxicity, for example hearing loss, can occur as a result of exposure to 35-40 Gy or higher, either as a single or cumulative dose. Radiation-induced gastrointestinal toxicity, which is similar to that occurring during chemotherapy, includes electrolyte loss, secondary infections, bloody diarrhea, and gastrointestinal bleeding, and can occur upon exposure to a radiation dose in the range from 5-20 Gy, or higher.

5. Loop Diuretics

Loop diuretics are a group of compounds with dissimilar chemical structure, but which share a similar mechanism and site of action within the kidney: these compounds inhibit sodium chloride reabsorption at the high-capacity site in the thick ascending limb of the loop of Henle, causing greatly increased excretion of sodium chloride in the urine, and to a lesser extent of potassium. Loop diuretics are among the compounds exhibiting the greatest diuretic effect, and are commonly used in the treatment of edema of cardiac, hepatic, or renal origin. Use of these compounds can cause ototoxicity at least in part as a result of the alteration of electrolyte composition in the inner ear. In adults, ototoxicity is generally reversible, disappearing upon withdrawal of the drug; however, permanent hearing loss has been reported, particularly with ethacrynic acid. In neonates, hearing loss may not be reversible and thus may be permanent. Commonly used loop diuretics include, but are not limited to, furosemide (Lasix and other compounds), ethacrynic acid (Edecrin), bumetanide (Bumex and other compounds), piretanide, muzolimine, indapamide (Lozol), and xipamide.

Loop diuretics greatly exacerbate the ototoxicity of platinum-containing antitumor compounds and aminoglycoside antibiotics. The interaction of these ototoxic compounds has been observed to be synergistic; the protectant combinations disclosed herein may prevent the ototoxicity of loop diuretics and/or their ototoxic synergistic interaction.

6. Iron Chelating Agents

Iron chelating agents such as deferoxamine mesylate (desferrioxamine mesylate; for example, Desferal) are used to treat patients exhibiting elevated levels of iron in the blood. Such patients include those suffering from sickle cell anemia, hereditary disorders resulting in elevated blood iron levels, those receiving frequent blood transfusions, those who have ingested large amounts of ferrous salts of iron (iron poisoning), etc. The use of iron chelating agents such as deferoxamine can result in ototoxicity.

7. Quinine and Quinidine

Quinine (Quinamm) has long been used as an antipyretic, analgesic, and antimalarial. Recently, it has been used to stabilize muscle membranes against repetitive activity. Specifically, it is used to treat myotonia congenita (Thomsen's disease) and nocturnal muscle cramp. Quinidine is a class IA antiarrhythmic agent useful in the treatment of atrial and ventricular arrhythmias.

The adverse side effects of quinine and quinidine are similar, and have been given the name “cinchonism,” deriving from the fact that quinine is obtained from the bark of the cinchona tree. These side effects include disturbances of hearing, including tinnitus, deafness, and vertigo.

8. Sudden Hearing Loss

Sudden hearing loss (SHL) is a medical emergency for which definitive diagnosis and treatment is still largely unknown. It was first described in the literature by De Klevn in 1944. SHL generally refers to hearing loss of sensorineural origin. It has been defined for research purposes and has been accepted by most authorities as 30 dB or more sensorineural hearing loss over at least three contiguous audiometric frequencies occurring within 3 days or less.

There are many potential causes of SHL, but despite extensive evaluation, the majority of cases elude definitive diagnosis and therefore, remain idiopathic. Reports estimate that the etiology of SHL is diagnosed in only 10% of cases. Suggested causes of idiopathic sudden sensorineural hearing loss (ISSNHL) include viral infections, immunologic, vascular compromise, and intracochlear membrane breaks. It is unlikely that any single one of these pathophysiologic processes explains all cases of ISSNHL. Treatment regimens aimed at addressing the underlying problem in each of these states have been suggested including decreasing cochlear inflammation, improving inner ear blood flow and oxygenation, and reestablishing the endocochlear potential.

The etiology of SHL can be broken down into broad categories: (1) viral and infectious, (2) autoimmune, (3) labyrinthine membrane rupture/traumatic, (4) vascular, (5) neurologic, and (6) neoplastic. There are multiple conditions within each of these categories that have been associated with sudden hearing loss.

9. Autoimmune Inner Ear Disorder

Autoimmune inner ear disease (AIED) is an inflammatory condition of the inner ear. It occurs when the body's immune system attacks cells in the inner ear that are mistaken for a virus or bacteria. AIED is a rare disease occurring in less than one percent of the 28 million Americans with a hearing loss.

The symptoms of AIED are sudden hearing loss in one ear progressing rapidly to the second ear. The hearing loss can progress over weeks or months. Patients may feel fullness in the ear and experience vertigo. In addition to vertigo, a ringing, hissing, or roaring sound in the ear may be experienced. Diagnosis of AIED is difficult and is often mistaken for otitis media until the patient develops a loss in the second ear.

Until recently it was thought that the inner ear could not be attacked by the immune system. Studies have shown that the perisacular tissue surrounding the endolymphatic sac contains the necessary components for an immunological reaction. The inner ear is also capable of producing an autoimmune response to sensitized cells that can enter the cochlea through the circulatory system.

C. Administration

The protectant combinations of the present invention can be administered orally or parenterally, for example intraperitoneally, by intravenous injection, intravenous infusion, etc., as described in Remington's Pharmaceutical Sciences, Fifteenth Edition, Mack Publishing Company, Easton, Pa., 1975. These combinations can also be given by local administration, for example, when a platinum-containing chemotherapeutic agent is administered by local injection, as noted above. Localized administration of protectant combinations can be carried out by topical application employing pharmaceutical formulations designed for this purpose as is known in the art, local injection, etc.

In one embodiment of the present invention, a protectant combination is administered topically to the round window membrane of the ear. Typically, such topical administration is carried out by applying a pharmaceutical formulation such as a topical solution comprising the protectant combination to the round window membrane by a micro-catheter or by injection into the middle ear. Suitable micro-catheters include those commercially available, for example, from IntraEar Corp., Denver, Colo. Preferably, the micro-catheter is attached to a battery-operated pump such as that commercially available from Disetronics, Inc., which is capable of automatically applying a topical solution comprising the otoprotective agent to the round window membrane, either continuously or intermittently.

Administration of the protectant combinations of the present invention simultaneously or contemporaneously with the administration of a platinum-containing chemotherapeutic agent, loop diuretic agent, aminoglycoside antibiotic, iron chelating agent, or quinine or quinidine, can be accomplished in several ways. For example, each agent can be formulated individually and separately administered simultaneously or sequentially via any of the routes described herein or which are otherwise conventional in the art. Alternatively, one or more protectant agents can be contained together in a single dose formulation that can be administered by a single route. As in the case of the platinum-containing chemotherapeutic agent, loop diuretic agent, etc., the dose of protectant combination can be administered in a single day.

The pharmaceutical compositions containing the protectant combinations of the present invention may be formulated in any conventional manner. Proper formulation is dependent upon the route of administration chosen. Pharmaceutically acceptable carriers for use in the compositions of the present invention are well known to those of ordinary skill in the art and are selected based upon a number of factors: the particular protectant combination used, and its concentration, stability and intended bioavailability; the disease, disorder or condition being treated with the composition; the subject, its age, size and general condition; and the route of administration. Suitable carriers are readily determined by one of ordinary skill in the art (see, for example, J. G. Nairn, in: Remington's Pharmaceutical Science (A. Gennaro, ed.), Mack Publishing Co., Easton, Pa., (1985), pp. 1492-1517, the contents of which are incorporated herein by reference).

The compositions are preferably formulated as tablets, dispersible powders, pills, capsules, gelcaps, caplets, gels, liposomes, granules, solutions, suspensions, emulsions, syrups, elixirs, troches, dragees, lozenges, or any other dosage form which can be administered orally. Techniques and compositions for making oral dosage forms useful in the present invention are described in the following references: 7 Modern Pharmaceutics, Chapters 9 and 10 (Banker & Rhodes, Editors, 1979); Lieberman et al., Pharmaceutical Dosage Forms: Tablets (1981); and Ansel, Introduction to Pharmaceutical Dosage Forms 2nd Edition (1976).

In alternative embodiments, the compositions can be formulated as a snack bar. In some of these embodiments, the snack bar is a conventional smack bar and typically includes a carbohydrate source, a protein source and optionally a fat source.

The compositions of the invention for oral administration comprise an effective protectant amount of a combination of the invention in a pharmaceutically acceptable carrier. Suitable carriers for solid dosage forms include sugars, starches, and other conventional substances including lactose, talc, sucrose, gelatin, carboxymethylcellulose, agar, mannitol, sorbitol, calcium phosphate, calcium carbonate, sodium carbonate, kaolin, alginic acid, acacia, corn starch, potato starch, sodium saccharin, magnesium carbonate, tragacanth, microcrystalline cellulose, colloidal silicon dioxide, croscarmellose sodium, talc, magnesium stearate, and stearic acid. Further, such solid dosage forms may be uncoated or may be coated by known techniques; e.g., to delay disintegration and absorption.

The protectant combinations of the present invention can also be formulated for parenteral administration, e.g., formulated for injection via intravenous, intraarterial, subcutaneous, rectal, subcutaneous, intramuscular, intraorbital, intracapsular, intraspinal, intraperitoneal, or intrasternal routes. The compositions of the invention for parenteral administration comprise an effective protective amount of the protectant combination in a pharmaceutically acceptable carrier. Dosage forms suitable for parenteral administration include solutions, suspensions, dispersions, emulsions or any other dosage form which can be administered parenterally. Techniques and compositions for making parenteral dosage forms are known in the art.

Additional minor components can be included in the combinations of the invention for a variety of purposes well known in the pharmaceutical industry. These components will for the most part impart properties which enhance retention of the protectant combination at the site of administration, protect the stability of the composition, control the pH, facilitate processing of the protectant combination into pharmaceutical formulations, and the like. Such components include cryoprotective agents for preventing reprecipitation of the protectants, surface active, wetting or emulsifying agents (e.g., lecithin, polysorbate-80, Tween® 80, pluronic 60, polyoxyethylene stearate), preservatives (e.g., ethyl-p-hydroxybenzoate), microbial preservatives (e.g., benzyl alcohol, phenol, m-cresol, chlorobutanol, sorbic acid, thimerosal and paraben), agents for adjusting pH or buffering agents (e.g., acids, bases, sodium acetate, sorbitan monolaurate), agents for adjusting osmolarity (e.g., glycerin), thickeners (e.g., aluminum monostearate, stearic acid, cetyl alcohol, stearyl alcohol, guar gum, methyl cellulose, hydroxypropylcellulose, tristearin, cetyl wax esters, polyethylene glycol), colorants, dyes, flow aids, non-volatile silicones (e.g., cyclomethicone), clays (e.g., bentonites), adhesives, bulking agents, flavorings, sweeteners, adsorbents, fillers (e.g., sugars such as lactose, sucrose, mannitol, or sorbitol, cellulose, or calcium phosphate), diluents (e.g., water, saline, electrolyte solutions), binders (e.g., starches such as maize starch, wheat starch, rice starch, or potato starch, gelatin, gum tragacanth, methyl cellulose, hydroxypropyl methylcellulose, sodium carboxymethyl cellulose, polyvinylpyrrolidone, sugars, polymers, acacia), disintegrating agents (e.g., starches such as maize starch, wheat starch, rice starch, potato starch, or carboxymethyl starch, cross-linked polyvinyl pyrrolidone, agar, alginic acid or a salt thereof such as sodium alginate, croscarmellose sodium or crospovidone), lubricants (e.g., silica, talc, stearic acid or salts thereof such as magnesium stearate, or polyethylene glycol), coating agents (e.g., concentrated sugar solutions including gum arabic, talc, polyvinyl pyrrolidone, carbopol gel, polyethylene glycol, or titanium dioxide), and protectants (e.g., sodium metabisulfite, sodium bisulfite, sodium sulfite, dextrose, phenols, and thiophenols). Preferably, each of these components is individually present in less than about 15 weight % of the total composition, more preferably less than about 5 weight %, and most preferably less than about 0.5 weight % of the total composition. Some components, such as fillers or diluents, can constitute up to 90 wt. % of the total composition, as is well known in the formulation art.

In some embodiments, the present invention can be prepared as a pharmaceutical suspension, comprising a protectant combination described above, a suspending agent, and a solvent. A suspension comprises a liquid vehicle, e.g., a pharmaceutically acceptable solvent, in which small particles of a solid, semisolid, or liquid are uniformly dispersed, but not dissolved. If the dispersion is not stable, the particles can be redispersed prior to administration by shaking, or stirring the mixture. In this embodiment, protectant agent particles are suspended using suspending agents noted below to produce a formulation with a protectant agent concentration that greatly exceeds the solubility of the compound. As used herein, the term “solvent” refers to aqueous solutions that may further comprise one or more co-solvents, such as alcohols (e.g., ethanol and propylene glycol), polyethylene glycols and their derivatives, glycerol and other body-tolerated solvents.

The concentration of protectant agent that can be prepared in the suspensions of the present invention can be readily determined by one of ordinary skill in the art using standard techniques and measurements. In general, the concentration of protectant agents in the suspensions of the present invention will be from about 20 mg/ml to about 2000 mg/ml. Suitably, the concentration will be about 100 mg/ml to about 1000 mg/ml, about 100 mg/ml to about 500 mg/ml, or about 200 mg/ml.

D-methionine, one of the protectant agents, has a solubility of about 5% (wt/wt) or approximately 50 mg/mL in water. Certain suspension formulations provide for a D-methionine concentration per unit volume of suspension of at least four times the solubility limit (a concentration of about 200 mg/ml).

As used herein, a “suspending agent” is any agent that can be used to generate a suspension of the protectant agents in a solvent system. Suitable suspending agents that can be used in the practice of the present invention include, but are not limited to sterically stabilizing substances such as poloxamers and poloxamines (block copolymers of polyoxyethylene and polyoxypropylene), ethoxylated esters of sorbitan fatty acids, including polysorbates (such as polysorbate 80 or Tween 80™), ethoxylated mono- and diglycerides, ethoxylated lipids, ethoxylated fatty alcohols, fatty acids and vitamin E-TPGS (d-alpha tocopheryl polyethylene glycol 1000 succinate). In suitable embodiments, the suspending agent may be a polysorbate, such as polysorbate 80. Appropriate concentrations of suspending agents for use in the practice of the present invention can be easily determined by those skilled in the art. The concentration of polysorbate 80 useful in preparing the suspensions of the present invention will be about 0.1 mg/ml to about 10 mg/ml, about 0.5 mg/ml to about 5 mg/ml or about 1 mg/ml.

In further embodiments, suspensions of the protectant combinations can further comprise preservatives. Any suitable preservatives known to those skilled in the art can be used in the suspensions of the present invention. For example, antimicrobial agents, antifungal agents, or antibacterial agents can be used. Suitable preservatives that may be used include, but are not limited to, butylated hydroxyanisole (BHA), butylated hydroxytoluene (BHT), benzyl alcohol, ethyl alcohol, parabens such as methyl-, ethyl-, propyl-, or butylparaben, chlorobutanol, sodium benzoate, benzoic acid, myristyl-gamma-picolinium chloride, benzalkonium chloride, benzethonium chloride, cetylpyridinium chloride, chlorocresol, cresol, dehydroacetic acid, methylparaben sodium, phenol, phenylethyl alcohol, potassium benzoate, potassium sorbate, propylparaben sodium, sodium dehydroacetate, sodium propionate, sorbic acid, thymol, and combinations thereof. Suitably, preservatives for use in the practice of the present invention will be parabens, such as methylparaben and propylparaben. Useful concentrations of such preservatives can routinely be determined by those skilled in the art. For example, methylparaben can be used in the protectant combination suspensions at a concentration of about 0.1 mg/ml to about 10 mg/ml, about 0.5 mg/ml to about 5 mg/ml or about 1 mg/ml, and propylparaben can be used at a concentration of about 0.01 mg/ml to about 1 mg/ml or about 0.05 mg/ml to about 0.5 mg/ml or about 0.1 mg/ml. Such preservatives can be used either alone or in combination with one another or other preservatives.

The protectant combination suspensions can also include one or more pharmaceutical excipients, such as thickening agents, humectants, sweetening agents and flavoring agents. Examples of thickening agents include, but are not limited to, carboxypolymethylene, sodium carboxymethylcellu lose, hydroxy-ethylcellu lose, xantan gum, hydroxypropylmethylcellulose, methylcellulose, and carrageenan. Examples of humectants include, but are not limited to, polyhydric alcohols, polyols such as glycerol, propylene glycol, propylene glycol glycerol, polyethylene glycol, isomalt, xylitol, maltitol, sorbitol, mannitol and the like. Examples of sweetening agents include, but are not limited to, sucrose, fructose, maltose, glucose and artificial sweeteners. Examples of flavoring agents include chocolate, thalmantin, aspartame, root beer, chewing gum, watermelon, cherry, orange, mango, or other flavorings stable at pH 7 to 9.

In various embodiments, a pharmaceutical suspension of a protectant combination of about 9 to 12 grams D-methionine, about 3 to 6 grams N-acetylcysteine or 3 to 6 grams acetylcarnitine 0.060 g methylparaben, 0.006 propylparaben, 0.072 g xantan gum, 0.060 g polysorbate 80, 3.0 g sorbitol, 0.1 ml flavor and up to 60 mL purified water in 60 mL total solution can be used.

D. Temporal Relationship of Dosing and Insult

In the various methods of the present invention, the effective amount of protective combination can be administered prior to, contemporaneously with, or subsequent to administration of the effective amount of platinum-containing chemotherapeutic agent, loop diuretic agent, aminoglycoside antibiotic, iron chelating agent, or quinine or quinidine, or exposure of the patient to harmful noise or radiation. Combinations of these time periods can also be employed. Generally, prior administration of the effective amount of the otoprotective agent can be conducted broadly within the period ranging from as much as 14 days (i.e., about 336 hours, about 168 hours, about 84 hours or about 60 hours or less) before administration of the platinum-containing chemotherapeutic agent, loop diuretic agent, etc., or exposure to noise or radiation. Likewise, subsequent administration of the effective amount of the otoprotective agent can be conducted broadly within the period including as much as 14 days (i.e., including about 60 hours, about 84 hours, about 168 hours or about 336 hours or more) after administration of the platinum-containing chemotherapeutic agent, loop diuretic agent, etc., or exposure to noise or radiation.

Preferably, prior administration of the effective amount of the methionine protective agent is within about 48 hours before administration of the platinum-containing chemotherapeutic agent, loop diuretic agent, etc., or exposure to noise or radiation; with subsequent administration within about 48 hours after administration of the platinum-containing chemotherapeutic agent, loop diuretic agent, etc., or exposure to noise or radiation. More preferably, prior administration can be within about 25 hours before, and subsequent administration can be within about 25 hours after, administration of the platinum-containing chemotherapeutic agent, loop diuretic agent, etc., or exposure to harmful levels of noise or radiation. Even more preferably, prior administration can be within about 6 hours before, and subsequent administration can be within about 1 hour after, administration of the platinum-containing chemotherapeutic agent, loop diuretic agent, etc., or exposure to noise or radiation. Even more preferably, prior administration of the effective amount of methionine protective agent can be within about 1 hour before, and subsequent administration can be within about 1 hour after, administration of the platinum-containing chemotherapeutic agent, loop diuretic agent, etc., or exposure to noise or radiation. Still more preferably, prior administration of the effective amount of methionine protective agent can be within about one-half hour before, and subsequent administration can be within about one-half hour after, administration of the platinum-containing chemotherapeutic agent, loop diuretic agent, etc., or exposure to noise or radiation.

### DEFINITIONS

As used herein, an “active ingredient” is an ingredient selected from a group consisting of methionine protectant agents of formula 1, N-acetylcysteine, carnitine, magnesium ions, lipoic acid, ebselen, glutathione, and glutathione ester.

As used herein, the term “ototoxicity” includes, but is not limited to, any detrimental or pathologic change in the structure or function of the ear, including changes in hearing and balance. Auditory functional changes can include, but are not limited to, hearing loss or other changes in auditory threshold for any stimulus, perception of sound including recruitment (abnormal growth in the perception of loudness), ability to identify, localize, recognize, distinguish between, or process sounds, and/or distortion of sounds or any abnormality as measured by conventional auditory tests. This term also includes tinnitus (ringing or noises in the ear), which includes any perception of sound that is not in response to an external signal. Further, ototoxicity includes any perceived or measured functional change in the balance or vestibular system, including, but not limited to, either induced or spontaneous vertigo, disequilibrium, increased susceptibility to motion sickness, nausea, vomiting, nystagmus, syncope, lightheadedness, dizziness, difficulty in visual tracking secondary to vestibular or balance disorder or abnormality as measured on any test of vestibular or balance function. Structural changes can include any intra- or extra-cellular, multicellular, or organ change in the auditory or vestibular pathways from the external ear up through and including the cortex and all pathways in between.

The term “otoprotective agent” refers to an agent that prevents, ameliorates, or otherwise protects against ototoxicity.

The term “neurotoxicity” includes, but is not limited to, any detrimental or pathologic change in the structure or function in the neurologic system or any part thereof. Neurologic functional changes can include, but are not limited to, neuropathy, either central or distal, including a common “stocking and glove” pattern, tingling, loss of sensation, numbness, decreased vibratory sensation, decreased deep tendon reflexes, sensory ataxia, neuritis, focal encephalopathy, aphasia, autonomic neuropathy, postural hypotension, a myasthenia-like syndrome, muscle cramps, headache, seizures, blindness or visual disturbance secondary to disorder of the optic or visual neurological pathway, papilledema, hearing loss secondary to disorder of the auditory neurologic pathway, and/or loss of the sensation of taste. Structural changes can include intra- or extra-cellular, multicellular, or organ changes, anywhere in the neurologic system, including both peripheral and central systems. Neurotoxicity can manifest itself during or after the course of treatment with platinum-containing anti-tumor compounds.

The term “neuroprotective agent” refers to an agent that prevents, ameliorates, or otherwise protects against neurotoxicity.

The term “gastrointestinal toxicity” includes, but is not limited to, any detrimental or pathologic change in the structure or function in the gastrointestinal system or any part thereof. Gastrointestinal changes include, for example, current or delayed nausea, vomiting, esophageal reflux, stomatitis, bleeding along the gastrointestinal tract, diarrhea, weight loss, and/or anorexia. Gastrointestinal toxicity can manifest itself during or after the course of treatment with platinum-containing anti-tumor compounds.

The term “gastrointestinal-protective agent” refers to an agent that prevents, ameliorates, or otherwise protects against gastrointestinal toxicity.

The lower alkyl groups described herein, either alone or containing the various substituents defined herein, can contain from one to six carbon atoms in the principal chain, and up to about 15 carbon atoms total. The lower alkyl groups include, for example, methyl, ethyl, propyl, isopropyl, butyl, hexyl, cyclopropyl, cyclopentyl, cyclohexyl, and the like. Substituents of the substituted alkyl groups described herein can include, for example, groups selected from alkyl, cycloalkyl, alkenyl, alkynyl, aryl, heteroaryl, O, S, N, P, or halogen (Cl, F, Br, or I) atoms. These substituent alkyl, cycloalkyl, etc., groups include, for example, lower alkoxy groups such as methoxy, ethoxy, and butoxy, and groups such as halo, nitro, amino, and keto.

The lower acyl groups described herein, either alone or containing the various substituents defined herein, can contain from one to six carbon atoms, and up to about 15 carbon atoms total. The lower acyl groups include, for example, an alkyl group of methyl, ethyl, propyl, isopropyl, butyl, hexyl, cyclopropyl, cyclopentyl, cyclohexyl, or an aryl group of phenyl or naphthyl. Substituents of the substituted acyl groups described herein can include, for example, groups selected from alkyl, cycloalkyl, alkenyl, alkynyl, aryl, heteroaryl, O, S, N, P, or halogen (Cl, F, Br, or I) atoms. These substituent alkyl, cycloalkyl, etc., groups include, for example, lower alkoxy groups such as methoxy, ethoxy, and butoxy, and groups such as halo, nitro, amino, and keto.

The alkenyl groups described herein, either alone or with the various substituents defined herein, are preferably lower alkenyl containing from two to six carbon atoms in the principal chain, and up to about 15 carbon atoms total. They can be substituted, straight, or branched chain, and include ethenyl, propenyl, isopropenyl, butenyl, isobutenyl, hexenyl, and the like.

The alkynyl groups described herein, either alone or with the various substituents defined herein, are preferably lower alkynyl containing from two to six carbon atoms in the principal chain, and up to about 15 carbon atoms total. They can be substituted, straight or branched chain, and include ethynyl, propynyl, butynyl, isobutynyl, hexynyl, and the like.

The aryl moieties described herein, either alone or with various substituents defined herein, can contain from about 6 to about 15 carbon atoms, and include phenyl. Substituents include alkanoxy, protected hydroxy, halogen, alkyl, aryl, alkenyl, acyl, acyloxy, nitro, amino, amido, etc. Phenyl is a preferred aryl.

The heteroaryl moieties described herein, either alone or with various substituents defined herein, can contain from about 5 to about 15 atoms, and include, furyl, thienyl, pyridyl and the like. Substituents include alkanoxy, protected hydroxy, halogen, alkyl, aryl, alkenyl, acyl, acyloxy, nitro, amino, and amido.

The acyloxy groups described herein can contain alkyl, cycloalkyl, alkenyl, alkynyl, aryl, or heteroaryl groups.

The carbon atoms, i.e., the methyl and methylene groups, constituting the principal backbone of the methionine or methionine-like moiety can also be substituted as variously described above.

Modifications and variations of the invention are possible without departing from the scope of the invention defined in the claims. Furthermore, all examples in the present disclosure are provided as non-limiting examples.

### EXAMPLES

### Example 1

### Test for Protective Effect of Protectant Combinations against Noise

An experimental study to evaluate the effect of administering protectant combinations to experimental animals that are exposed to noise sufficient to induce ototoxicity is described. The design of this study comprises three independent groups of subjects, where one group is used as the control and the other groups receive the treatment. The subjects are male adult chinchilla laniger divided equally into three experimental groups. The three groups consist of a saline control group, a pre-noise protectant combination treatment group, and a post-noise protectant combination treatment group. Baseline hearing thresholds obtained through auditory brainstem response (ABR) are taken before the initial noise exposure and at 21 days after noise exposure for each subject.

ABR testing is performed using an Intelligent Hearing Systems evoked potential unit. Subcutaneous electrodes are placed at the vertex (non-inverting), to a point directly below the ipsilateral pinna (inverting) with a ground electrode in the hind leg. For measurement of the ABR threshold, animals are fully anesthetized throughout all ABR procedures with 1 ml/mg intramuscular injection of Rompun cocktail, which is a solution containing 86.21 mg/kg ketamine and 2.76 mg/ml Xylazine. This initial injection is supplemented as needed with half doses. Stimuli consisted of tone bursts (1 ms Blackman rise/fall ramp, 0 ms plateau) at octave intervals 2, 4, 6, and 8 kHz. All acoustic stimuli are routed through a computer-controlled attenuator to an insert earphone (Etymotic Research). The sound delivery tube of the insert earphone is positioned approximately 5 mm from the tympanic membrane. Earphone sound delivery is calibrated using a coupler attached to a sound level meter approximating the distance from the earphone to the tympanic membrane. Five hundred samples are collected from the recording electrode, at a rate of 10/s. An intensity series is obtained for each animal starting at 100 dB SPL and then in 10-dB descending steps. Threshold is defined as the lowest level eliciting a replicable, visually detectable response. All ABR testing is conducted in a double walled sound booth.

Prior to being exposed to noise, the animals are housed in the Southern Illinois University School of Medicine Laboratory Animal Care Facility (LAM) for a minimum of 5 days. The animals are acclimated to the wire cages and sound exposure booth prior to the noise exposure. All noise exposure is administered inside a sound booth housed in LAM. The sound booth isolates the noise exposures from the outside environment. This noise exposure protocol is developed from the procedure of Hu et al., Hear. Res. 113:198-206 (1997). Specifically, an octave band noise centered at 4 kHz is generated by a TDT GNS 40X white noise generator routed through an attenuator (TDT PA3), a filter (Krohn-Hite 3384) and a power amplifier (Sony 55ES) to a custom built acoustic exponential horn with a maximum output at 4kHz using an Altec 290E driver. The loudspeaker is suspended directly above the cage with the nozzle feeding into the cage, giving the animals access to water during the noise exposure period. Each animal is exposed to noise at a level of 105 dB SPL for 6 hours. During the noise exposure, the animal is unrestrained in a small wire cage with ad-lib water access. When the animals are not being exposed to noise, they are housed in a quiet animal colony.

Animals receive one dose of a protectant combination (comprising two or more of methionine protectant agents, carnitine, N-acetylcysteine, magnesium ions, lipoic acid, ebselen, glutathione, and glutathione ester) or saline by intraperitoneal injection starting (1) 30 minutes pre noise exposure and (2) 1 hour post noise exposure for one dose plus 4 additional doses BID (5 doses). Auditory threshold shifts are calculated as post-noise thresholds (dB SPL) minus baseline threshold (dB SPL). Means are plotted as a function of treatment group (saline-noise and protectant combination post-treatment), over time (zero, 21 days) and by threshold test frequency. The statistical analysis consists of Wilcoxon's rank sum test on the difference scores between the two time points, for each of the four frequencies separately. The analysis is performed twice to examine the effect, if any, of the use of both ears from the same subject in a subset of the data. Thus, the sample size is 10 subjects per group in each analysis.

### Example 2

### Test for Protective Effect of Protectant Combinations against CDDP

Animals. As is well known to those of ordinary skill in the art, the rat is a well-accepted experimental animal useful as a model for studies of CDDP toxicity in humans.

Complete data sets are obtained for five groups of five male Wistar rats (280-421 g). All animals are anesthetized with 1 ml/mg IM of Rompun cocktail (a solution containing 86.21 mg/ml ketamine and 2.76 mg/ml xylazine) prior to all injections and testing. Anesthesia is supplemented as needed with half doses throughout testing. The five groups include: a treated control group which received a dose of CDDP dissolved in normal sterile saline (1 mg of CDDP/ml normal saline; solution pH 6.3) administered by i.p. infusion with a Harvard Apparatus Infusion Pump, over a 30 minute period, an untreated control group that received an equivalent volume of normal saline (pH 6.5) instead of CDDP, and three experimental groups that receive various doses of protectant combination (comprising two or more of methionine protectant agents, carnitine, N-acetylcysteine, magnesium ions, lipoic acid, ebselen, glutathione, and glutathione ester) within the ranges described below dissolved in 3-5 ml of normal saline (solution pH 6.6) delivered by slow (over 1-2 minutes) i.p. injection 30 minutes prior to the same CDDP infusion as the treated control group. Both CDDP (purchased from Sigma Chemical Co., St. Louis) and the protectant combinations are freshly prepared before each experiment. For the treated control group, a total of 10 animals are needed to obtain 5 animals with complete data sets because 50% of the animals may not survive to the end of the study period. Only 5 animals are needed in the untreated control and in each of the D-Met pretreated groups because all of the animals in each of these groups are expected to survive until the end of the study period.

Evoked Potentials. Auditory Brainstem Testing (ABR) is used to assess auditory threshold. Testing occurs just prior to administration of the CDDP or saline (with or without a protective combination) and again 3 days later. All testing is performed with the animal in a double walled IAC booth.

Platinum/iridium needle electrodes are placed at the vertex (non-inverting) to a point directly below the ipsilateral pinna (inverting) with a ground electrode placed in the hind leg.

ABR data collection is obtained with a Biologic Traveler system with an additional custom made high frequency stimulator for 14000 Hz. ABR thresholds are measured in response to 100 microsecond clicks and for tonebursts with 1 ms rise/fall and 0 ms plateau gated by a Blackman envelope and centered at the frequencies of 1, 4, 8, and 14 kHz presented at 10/s. An intensity series is obtained for each animal from 100 to 0 dB peak equivalent SPL (peSPL) for click stimuli and Sound Pressure Level (SPL) for tonebursts in 10 dB decrements. The term peSPL means that the amplitude of the click stimulus from the prestimulus baseline to the first peak is equivalent to the SPL of a pure tone stimulus having the same prestimulus baseline to peak amplitude. Threshold is defined as the lowest intensity capable of eliciting a replicable, visually detectable response.

A total of 512 sweeps constitutes each average. The recording epoch is 15 ms following stimulus onset. Responses are analogue filtered with a 30-3000 Hz bandpass.

Rectal temperature is monitored throughout recordings, with animal temperature being maintained by a warming pad.

Electron microscopy. The animals are sacrificed by decapitation while under general anesthesia and cochleae perfused with fixative through the perilymphatic spaces. The primary fixative is 2.5% glutaraldehyde at 4 C in 0.1 M phosphate buffer (pH 7.4). A small hole in the otic capsule is hand drilled beneath the first turn with a three sided, sharpened pick. In vitro perfusion is performed intermittently within 5 minutes of sacrifice through the small hole in scala tympani, allowing the fluid to exit through the opened oval window. After perfusion fixation, the round window membrane is removed, and the cochleae are immersed in glutaraldehyde and stored in the refrigerator overnight.

After overnight fixation in glutaraldehyde, the cochleae are rinsed in 0.1 M phosphate buffer and gently perfused with the buffer through the perilymphatic spaces by loosely fitting the tube end of the perfusion syringe over the opening drilled in the scala tympani. Cochleae are then rinsed in buffer 3 times. After rinsing, the cochleae are post-fixed by a perfusion of 1.5% OsO₄ (at 4° C.) in phosphate buffer in a fume hood. Fixation is continued by immersion and rotation in the same fixative for 15 minutes. The cochleae are rinsed in the same fashion as after glutaraldehyde fixation.

Under the dissecting microscope, the bony capsule of the cochlea is carefully removed.

The tissue is then serially dehydrated in 2×50%, 70%, 85%, 95% and 3×100% ethanol. Each specimen is dried using Peldri and placed on a stub for sputter coating with 13 nm platinum. The tissue is viewed through a Hitachi S-500 scanning electron microscope and photographs taken on Polaroid type 55 land Film.

Semi-quantitative analysis per turn for the outer hair cells is performed in the following manner. For each turn of the cochlea, apical, middle, and base, a representative sample is examined. For each sample, 11 inner hair cells serve as a guide to count a section of 33 outer hair cells or 11 per row. The number of damaged or missing outer hair cells within each sample is then counted.

Weight. Each animal's weight is measured in an Ohaus triple beam balance scale before administration of the anesthetic for the pretest and again before the post-test 3 days later.

Statistical Analysis. ABR data are analyzed using a three factor analysis of variance (ANOVA) with one between subject factor (groups) and two within subject factors (frequency and pre- vs. post-test). Each dependent variable is analyzed independently. Tests subsequent to the ANOVA are carried out in accordance with the Tukey HSD procedure. Weight loss and/or gastrointestinal protection is measured using the same type of statistical analysis as the ABR measures. SEM data are analyzed for each turn using a one way analysis of variance with Post-Hoc Tukey HSD analysis. The criterion for statistical significance for all measures is p 0.01.

### Example 3

### Test for Protective Effect of Protectant Combinations against Amikacin

The experiments are administered with fifteen male Hartley white guinea pigs (250-350 grams) comprising three groups of 5 animals each. Each group receives a daily injection for 28 days according to the following protocol: (1) a treated control group receives 200 mg/kg/day amikacin; (2) an untreated control group receives an equivalent volume saline injection; and (3) an experimental group receives 300 mg/kg/day protectant combination 30 minutes prior to administration of 200 mg/kg/day amikacin.

Auditory brainstem response testing (ABR) is performed immediately prior to the first injection and again at 28 days, just prior to sacrifice. Subcutaneous electrodes are placed at the vertex (non-inverting), to a point directly below the ipsilateral pinna (inverting) with a ground electrode in the hind leg.

ABR data collection is obtained with a Biologic Navigator System with an additional custom-made high frequency stimulator for 14 kHz. ABR thresholds are measured in response to 100 microsecond clicks and for tone-bursts with 2 millisecond rise/fall for 1 kHz, and 1 millisecond rise/fall for 4, 8 and 14 kHz and 0 millisecond plateau gated by a Blackman envelope and centered at the frequencies of 1, 4, 8 and 14 kHz presented at 10/s. An intensity series is obtained for each animal from 100 to 0 dB peak equivalent sound pressure level (peSPL) for click stimuli and sound pressure level (SPL) for tone-bursts in 10 dB decrements. The term peSPL means that the amplitude of the click stimulus from the pre-stimulus baseline to the first peak is equivalent to the SPL of a pure tone stimulus having the same pre-stimulus baseline to peak amplitude. Threshold is defined as the lowest intensity capable of eliciting a replicable, visually detectable response.

A total of 512 sweeps constitutes each average. The recording epochs are 15 ms following stimulus onset. Responses are analog filtered with a 30-3000Hz bandpass.

Rectal temperatures are monitored throughout recordings with animal temperature maintained by a warming pad.

All animals are fully anesthetized throughout all ABR testing, and prior to sacrifice with a 1 mL/kg IM injection of Rompun cocktail (a solution containing 86.21 mg/mL ketamine and 2.76 mg/mL xylazine) which is supplemented as needed with half doses.

All procedures are approved by the SIU School of Medicine animal care and use committee, and are in compliance with “The Principles of Laboratory Animal Care” formulated by the National Society for Medical Research and the “Guide for the Care and Use of Laboratory Animals” prepared by the National Academy of Sciences and published by the National Institutes of Health (NIH publication No. 80-23, revised 1978).

In some of the various embodiments, the useful protectant combination doses for Examples 1 to 3 are: (1) 100 mg/kg to 250 mg/kg methionine and 50 mg/kg to 150 mg/kg acetylcarnitine, preferably in a 2:1 ratio of methionine to acetylcarnitine; (2) 100 mg/kg to 250 mg/kg methionine and 40 mg/kg to 150 mg/kg N-acetylcysteine, preferably in a 2:1 ratio of methionine to N-acetylcysteine; (3) 100 mg/kg to 250 mg/kg methionine and 30 mg/kg to 80 mg/kg magnesium ions, preferably in a 2:1 ratio of methionine to magnesium ions; (4) 100 mg/kg to 250 mg/kg methionine and 0.2 mg/kg to 3 mg/kg lipoic acid, preferably in a 2:1 ratio of methionine to lipoic acid; (5) 100 mg/kg to 250 mg/kg methionine and 20 mg/kg to 100 mg/kg ebselen, preferably in a 2:1 ratio of methionine to ebselen; (6) 50 mg/kg to 150 mg/kg acetylcarnitine and 40 mg/kg to 150 mg/kg N-acetylcysteine; (7) 50 mg/kg to 150 mg/kg acetylcarnitine and 30 mg/kg to 80 mg/kg magnesium ions; (8) 50 mg/kg to 150 mg/kg acetylcarnitine and 0.2 mg/kg to 3 mg/kg lipoic acid; (9) 40 mg/kg to 150 mg/kg N-acetylcysteine and 30 mg/kg to 80 mg/kg magnesium ions; and (10) mg/kg to 150 mg/kg N-acetylcysteine and 0.2 mg/kg to 3 mg/kg lipoic acid. The combinations or compositions detailed in Table 1 could also be used in the experimental protocols of Examples 1 to 3.

What is claimed is:

1. A therapeutic combination comprising two or more protectant agents selected from a group consisting of methionine protectant agents, N-acetylcysteine, carnitine, magnesium ions, lipoic acid, ebselen, glutathione, and glutathione ester;
wherein each of said methionine protectant agents corresponds to Formula 1 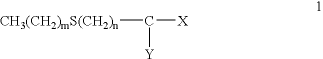
wherein m is an integer from 0 to 3; n is an integer from 1 to 3; X═—OR¹, —OCOR¹, —COOR¹, —CHO, —CH(OR¹)₂, or —CH₂OH; Y═—NR²R³ or —OH; R¹═H or a substituted or unsubstituted, straight branched chain, or cyclic alkyl group having 1 to 6 carbon atoms; R²═H or a substituted or unsubstituted, straight or branched chain acyl group having 1 to 6 carbon atoms; and R³═H or a substituted or unsubstituted, straight or branched chain acyl group having 1 to 6 carbon atoms; or a pharmaceutically acceptable salt thereof;
wherein at least one of said protectant agents is present in a concentration of at least about 10 wt. % based on the total weight of active ingredients.

2. The combination of claim 1 comprising two or more methionine protectant agents.

3. The combination of claim 2 wherein the combination of protectant agents consists only of methionine protectant agents.

4. The combination of claim 1 comprising a combination of two of said protectant agents other than glutathione.

5. The combination of claim 1 wherein at least one of said protectant agents comprises a methionine protectant agent.

6. The combination of claim 1 wherein the combination comprises a methionine protectant agent and two or more other protectant agents selected from the group consisting of N-acetylcysteine, carnitine, magnesium ions, lipoic acid, ebselen, glutathione, and glutathione ester.

7. The combination of claim 1 comprising a methionine protectant agent and N-acetylcysteine wherein said methionine protectant agent is present in a concentration of at least about 10 wt. % based on the total weight of active ingredients.

8. The combination of claim 7 further comprising carnitine.

9. The combination of claim 1 comprising a methionine protectant agent and carnitine wherein said methionine protectant agent is present in a concentration of at least about 10 wt. % based on the total weight of active ingredients.

10. The combination of claim 1 comprising a methionine protectant agent and magnesium ions wherein said methionine protectant agent is present in a concentration of at least about 10 wt. % based on the total weight of active ingredients.

11. The combination of claim 1 comprising a methionine protectant agent and lipoic acid wherein said methionine protectant agent is present in a concentration of at least about 10 wt. % based on the total weight of active ingredients.

12. The combination of claim 1 comprising a methionine protectant agent and ebselen wherein said methionine protectant agent is present in a concentration of at least about 10 wt. % based on the total weight of active ingredients.

13. A therapeutic composition of claim 1 comprising two or more protectant agents selected from a group consisting of methionine protectant agents, N-acetylcysteine, carnitine, magnesium ions, lipoic acid, ebselen, glutathione, and glutathione ester;
wherein said methionine protectant agent corresponds to Formula 1

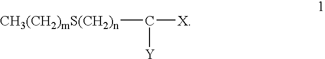

14. The composition of claim 13 comprising a methionine protectant agent and N-acetylcysteine wherein said methionine protectant agent is present in a concentration of at least about 10 wt. % based on the total weight of active ingredients.

15. The composition of claim 14 further comprising carnitine.

16. The composition of claim 13 comprising a methionine protectant agent and carnitine wherein said methionine protectant agent is present in a concentration of at least about 10 wt. % based on the total weight of active ingredients.

17. A method for treating
(a) ototoxicity in a patient exposed to noise for a time and at an intensity sufficient to result in ototoxicity;
(b) ototoxicity, nephrotoxicity, neurotoxicity, alopecia, gastrointestinal disorder, or reduced survival in a patient undergoing treatment with a chemotherapeutic effective amount of an anti-tumor platinum-coordination compound;
(c) ototoxicity in a patient undergoing treatment with an aminoglycoside antibiotic;
(d) ototoxicity, neurotoxicity, alopecia, gastrointestinal disorder, or reduced survival in a patient exposed to radiation for a time and at an intensity sufficient to result in ototoxicity, neurotoxicity, alopecia, gastrointestinal disorder, or reduced survival;
(e) sudden hearing loss in a patient; and
(f) autoimmune inner ear disease in a patient;
the method comprising administering to said patient an effective amount of a protectant combination of claim 1.

18. The method of claim 17 wherein said protectant combination is a composition.

19. A method for treating sudden hearing loss in a patient comprising administering to said patient an effective amount of a protectant agent comprising a methionine protectant agent, said methionine protectant agent corresponding to Formula 1 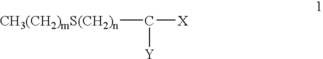
wherein m is an integer from 0 to 3; n is an integer from 1 to 3; X═—OR¹, —OCOR¹, —COOR¹, —CHO, —CH(OR¹)₂, or —CH₂OH; Y═—NR²R³ or —OH; R¹═H or a substituted or unsubstituted, straight, branched chain, or cyclic alkyl group having 1 to 6 carbon atoms; R =H or a substituted or unsubstituted, straight or branched chain acyl group having 1 to 6 carbon atoms; and R³═H or a substituted or unsubstituted, straight or branched chain acyl group having 1 to 6 carbon atoms; or a pharmaceutically acceptable salt thereof.

20. A method for treating autoimmune inner ear disease in a patient comprising administering to said patient an effective amount of a protectant agent comprising a methionine protectant agent, said methionine protectant agent corresponding to Formula 1 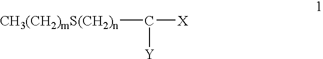
wherein m is an integer from 0 to 3; n is an integer from 1 to 3; X═—OR¹, —OCOR¹, —COOR¹, —CHO, —CH(OR¹)₂, or —CH₂OH; Y═—NR²R³ or —OH; R¹═H or a substituted or unsubstituted, straight branched chain, or cyclic alkyl group having 1 to 6 carbon atoms, preferably 1 to 4 carbon atoms; R²═H or a substituted or unsubstituted, straight or branched chain acyl group having 1 to 6 carbon atoms, preferably 1 to 4 carbon atoms; and R³═H or a substituted or unsubstituted, straight or branched chain acyl group having 1 to 6 carbon atoms, preferably 1 to 4 carbon atoms; or a pharmaceutically acceptable salt thereof.
